(19) United States
(12) Patent Application Publication
(10) Pub. No.: US 2026/0033748 A1
(43) Pub. Date: Feb. 5, 2026

(54) HEARING ABILITIES ASSESSMENT

(71) Applicant: The Court of Edinburgh Napier University, Edinburgh (GB)

(72) Inventor: Iain MCGREGOR, Edinburgh (GB)

(73) Assignee: The Court of Edinburgh Napier University, Edinburgh (GB)

(21) Appl. No.: 19/099,585

(22) PCT Filed: Jul. 20, 2023
(86) PCT No.: PCT/EP2023/070226

§ 371 Date: Jan. 29, 2025

(30) Foreign Application Priority Data
Aug. 25, 2022 (GB) 2212381.4

(51) Int. Cl. A61B 5/12; A61B 5/00; A61B 5/11

(52) U.S. Cl. CPC A61B 5/123; A61B 5/0077; A61B 5/1114; A61B 5/1128; A61B 5/4064; A61B 5/6803; A61B 2562/0219

(57) ABSTRACT

A method for assessing hearing abilities of a human person having two cars, the method comprising: —providing two audio signals (S₁, S₂) to two respective car speakers (15, 16) at a person's corresponding cars; —measuring at least one value of at least one parameter (x₁₅, y₁₅, z₁₅, x₁₆, y₁₆, z₁₆) representative of the person's head position, adapting at least one of the two audio signals (S₁, S₂) based on the at least one measured value ((x₁₅, y₁₅, z₁₅, x₁₆, y₁₆, z₁₆), and repeating the measuring and adapting often enough for the sounds provided to the person to correspond to their actual head position; —detecting that the person has found their best perception of the two sounds; —calculating from the person's head position corresponding to the best perception an overall sensitivity value, a differential sensitivity value and/or a value of a latency shift of the person.

The invention relates to audiometry, more precisely to subjective audiometry, that is to individual's hearing abilities assessment based on the individual responses.

While objective audiometry relies on physical measurements, subjective audiometry depends on the responses of the person whose hearing abilities are assessed.

Classically, a single frequency tone with decreasing amplitude is presented to an ear, and when the user no longer hears anything, the user mentions it and the corresponding amplitude value is stored as a threshold for this frequency. Repeating this for multiple specified frequencies in each ear facilitates the creation of an audiogram.

It may also be tested through the person's ability to detect and repeat spoken words heard at varying volumes through a headset (speech audiometry).

The traditional audiogram may be affected by variations in reproduction hardware, levels of listening (Equal Loudness contours/Fletcher Munson curves) or variations in the noise floor of auditory environments.

Further, subjective audiometry relies upon subjective responses.

There is actually a need for a more accurate subjective hearing abilities assessment.

There is provided a method for assessing hearing abilities of a human person having two ears, the method comprising:
(a) providing two ear speakers to the person, one for each ear;
(b) for each ear speaker, providing an audio signal to the ear speaker for the ear speaker to convert said audio signal into a sound at the person's corresponding ear;
(c) measuring at least one value of at least one parameter representative of the person's head position, adapting at least one of the two audio signals based on the at least one measured value, and repeating the measuring and adapting often enough for the sounds provided to the person to correspond to their actual head position;
(d) detecting that the person has found their best perception of the two sounds, e.g., that the two sounds are perceived with equal amplitudes,
(e) responsive to the detection, storing at least one current value of at least one parameter representative of the person's head position;
(f) calculating from the at least one stored value of the at least one parameter representative of the person's head position, an overall sensitivity value, a differential sensitivity value and/or a value of a latency shift, e.g., an internal ear phase shift, of the person.

That is, sounds are provided at the person's ears. The person may naturally move their head to achieve the best perception as the sound(s) vary(ies) with their head position. When this best perception is achieved, e.g., when the person does not significantly move their head for a lapse of time, the overall sensitivity, differential sensitivity and/or latency shift value(s) may be calculated from this current, best perception, head's position.

Measurement is not triggered by a decision the person has to make while the amplitude of the sound is decreasing or increasing, as in the prior art, but by the person no longer moving and/or indicating that their perception is as good as can be, e.g. that this is the quietest sound the person can hear, that sounds perceived at the left and right ear have same perceived volume, etc. The assessment may thus be more accurate and reliable, since the person has time to find out the position that will correspond to the best perception.

Further, the invention is not limited to specific audio signals, thus allowing a better assessment of hearing sensitivity. Further,it can take into consideration the listening conditions. For example, multiple profiles may be created for a single user, which can (advantageously) then be automatically selected as the auditory environment changes. In an embodiment, the profiles may also vary depending on the type of audio signal to be provided to the person, e.g., if the audio signal corresponds to speech the audiogram and/or correction factors values may be different than if the audio signal corresponds to music.

Improved granularity may also be achieved.

Further, this method may allow the detection and possibly the correction of phase shifts due to physical anatomy or other factors.

The invention is not limited to a particular type of ear speaker, as long as it can be placed on or worn in the ear and converts an audio signal to a sound to be listened at this ear. Each ear speaker thus receives a corresponding audio signal and outputs a sound at the user's ear.

The two ear speakers may for example be part of a headset, a head mounted display (HMD), or a headphone. Alternatively, each ear speaker may comprise an earbud, an earpiece, a telephone, etc. A mobile phone mounted into a virtual reality viewer, e.g., a cardboard viewer such as Google cardboard, could be also used, in particular if it has twin front-facing speakers.

The audio signal is a representation of sound that may for example comprise an analogue signal or a digital signal. The audio signal may for example be an electric signal or an optical signal.

The audio signal may have frequency(ies) within the audible range (roughly from 20 Hz to 20 kHz).

The audio signal may be synthesized. Alternatively, it may originate from a sound recording, e.g., by a microphone.

The term “head position” should not be interpreted as a synonym of a position of the barycentre of the head. It may typically encompass the orientation of the head as it is related the locations of the ears.

For example, the at least one parameter may comprise an azimuthal angle representing the orientation of the head. In another example, the at least one parameter may further comprise a polar angle.

Of course, the at least one parameter may comprise a location of a determined point or segment of the head, e.g., an ear or a segment between the two ears.

In an embodiment, the at least one parameter may comprise two solid angles, e.g., one for each ear.

In an embodiment, the at least one parameter may comprise two sets of coordinates, one for each ear, e.g., Cartesian coordinates or spherical coordinates.

The parameter representative of the head position may in particular be representative of a rotation of the head (and thus, of the pinnae).

This at least one parameter may represent a translation of the head, in particular if the at least one parameter representative of the head position impacts the phase of at least one audio signal. It may comprise a displacement vector, a length, a length and an angle, etc.

Of course the at least one parameter may represent both a translation of the head and a rotation of the head.

The invention is thus not limited to a particular type of parameter, as long as it represents the head position. The at least one parameter may for example comprise Cartesian coordinates, spherical coordinates, a pair of angles values comprising an azimuthal angle value and a polar angle value, a single angle value, etc.

The at least one value of the at least one parameter representative of the person head position results from measurement(s). For example, the position of the ear speakers may be measured by means of sensor(s) integrated into the ear speakers, e.g., by means of two gyroscopes or microelectromechanical systems (MEMS) accelerometers integrated within the respective ear speakers. In an alternative embodiment, at least one outside sensor may be provided, e.g., on the head, to monitor the positions of the ears. The outside sensor(s) may be part of a head-mounted display (HMD). In another embodiment, one or more cameras, e.g., a webcam or a smartphone camera, may be provided to capture video(s) of the person, and the video(s) may be analysed to track the motion of the head and/or the positions of the ears, in the three-dimensional environment.

Once the ears and/or head position(s) is/are measured, at least one of the two audio signals is recalculated based on the most recent values of the head position.

These measurements and the subsequent adaptations of the audio signal(s) may be repeated often enough, e.g., at regular time intervals, for the sound signals issued from a conversion of the audio signals to correspond to the current position of the head, as it is perceived by human persons. For example, the head position may be measured at a frequency higher than 1 s⁻¹, preferably higher than 10 s⁻¹, advantageously higher than 100 s⁻¹, e.g., every millisecond. The sound(s) output at the ears of the person thus vary(ies) in real time with the position of the head.

For example, the at least one parameter representative of the person's head position may comprise at least a first and a second parameters, such as for example:
Cartesian coordinates, e.g. {x, y}, {x, z}, {y, z}, or {x, y, z},
Spherical coordinates, e.g., {r, θ, φ}, {r, θ}, {r , φ} or {θ, φ}.
A parameter representing a displacement, e.g., a projection of a displacement in a horizontal plane parallel to the floor, and a parameter representing a rotation of the head;
Etc.

In an embodiment, repeatedly adapting the at least one of the two audio signals based on the at least one measured value may comprise repeatedly adapting a first parameter, e.g. an amplitude, of the at least one of the two audio signals based on the at least one measured value of the first parameter representative of the head position, and repeatedly adapting a second parameter of the at least one of the two audio signals distinct from the first parameter, e.g., a phase shift between the two audio signals, based on the at least one measured value of the second parameter representative of the head position. This may allow obtaining audiogram data more efficiently. Indeed, when a position of best perception is achieved, e.g., when the person no longer moves their head, more data may be obtained, e.g., not only a differential sensitivity value but also a latency shift and/or an overall sensitivity.

For example, x parameter (e.g., indicating movements at the left of the right of the person) may be associated to frequency correction or relative amplitude, y parameter (e.g., corresponding to forward and backward movements) to phase shift, and z (e.g., corresponding to upward and downward movements) to overall volume.

The person may naturally perceive the adaptations of the audio signal for various directions/movements and move their head to a position providing best perception regarding at least two criteria, e.g., equal loudness and clarity.

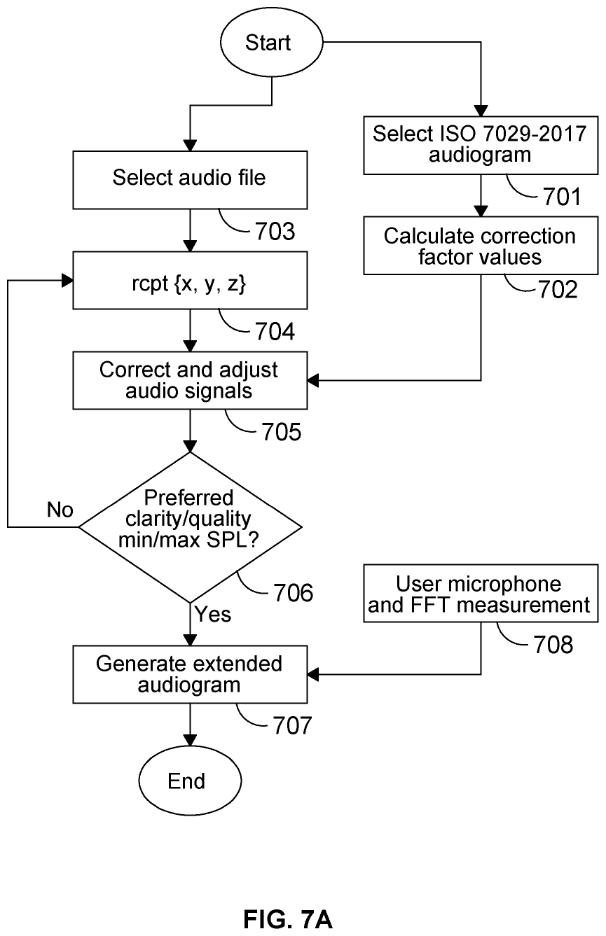

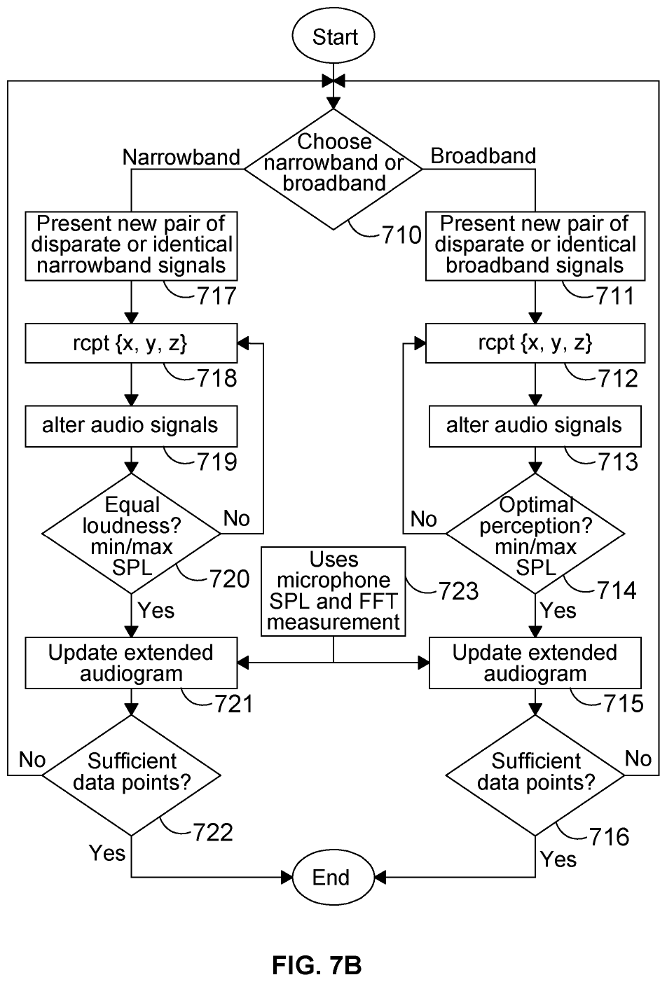

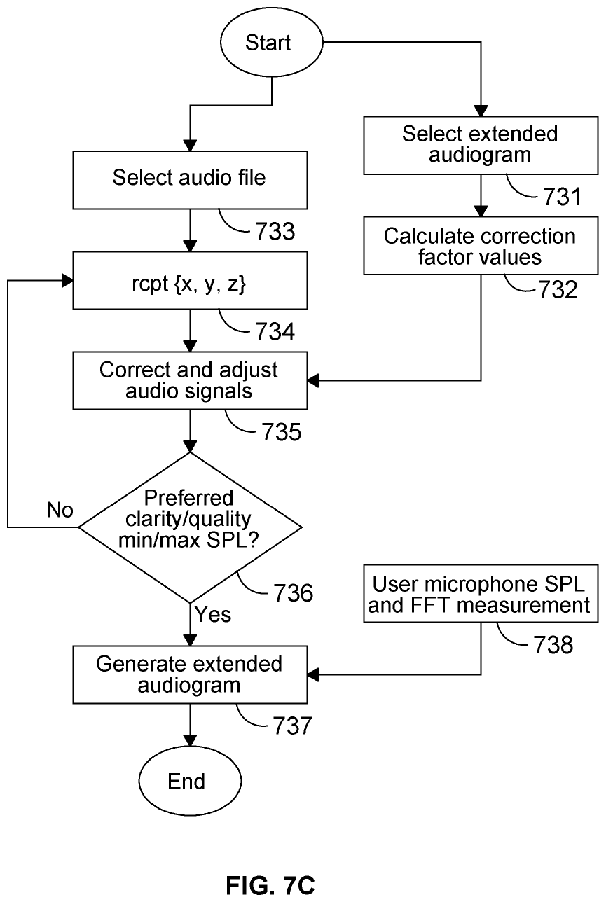

Example of audio signals parameters that may be adapted may comprise an amplitude of one only of the audio signals, an amplitude of both audio signals, a phase shift between the audio signals, a frequency correction based on another audiogram (e.g., a standard audiogram as illustrated by FIG. 7A or a previously calculated audiogram as illustrated by FIG. 7C), etc.

For example, the first and second parameter may comprise, respectively:
A frequency correction and a relative amplitude;
A frequency correction and a phase shift between the two audio signals;
A relative amplitude and an overall amplitude,
Etc.

Many associations between parameters representative of the person's head position and audio signals parameters are possible. For example, according to an example embodiment:
Azimuthal angle θ value affects relative amplitude between audio signals, e.g., when the person rotates to its left, the audio signal for the left ear gets louder than it was, and when the person rotates to its right, the audio signal for the left ear becomes quieter than it was;
Elevation φ value affects overall amplitude of both audio signals, e.g., when the head goes down, both audio signals become quieter and when the head goes up, both audio signals become louder; and/or
Depth value r affects phase, e.g., if the head moves forward, the audio signal for the right ear may be shifted ahead as compared to the signal for the left ear, and if the head moves backward, the audio signal for the right ear may be delayed as compared to the signal for the left ear.

Advantageously, the at least one parameter representative of the person's head position may comprise a third parameter, such as a projection of a displacement along a vertical axis parallel to the gravity vector or another parameter.

Advantageously, the first, second and third parameters may form coordinates of a coordinate system, such as a Cartesian coordinates system.

In an embodiment, repeatedly adapting the at least one of the two audio signals based on the at least one measured value comprising repeatedly adapting a third parameter of the at least one of the two audio signals based on the at least one measured value of the third parameter, the third parameter being distinct from the first and second parameter. Thus, the method leverages movements within the 3 dimensions of space to obtain more data.

In an embodiment, when the assessment starts, the audio signals may already depend on at least one parameter representative of the person's head position. That is, the at least one parameter representative of the person's head position is previously measured and at least one of the audio signals is calculated accordingly. For example, the audio signals may correspond to a virtual source having a predetermined position in the environment.

Alternatively, an initial audio signal may be independent on the head position. Then it is monitored how the person moves their head and at least one of the audio signals is adapted accordingly. For example, the initial audio signals may correspond to a virtual source having a determined angle relative to the head initial position; the person may thus rotate their head, and the angle corresponding to the best perception (which is expected to differ at least slightly from the angle of the virtual source in the initial audio signals as no human person has perfect hearing abilities) is stored and used to calculate sensitivity.

The method comprises detecting that the person has found their best perception of the two sounds. Optimized perception may for example comprise perceiving the sounds at their two ears with equal amplitudes. Alternatively or additionally, it may be detected that the person perceives the two sounds as originating from a single simulated spatially limited acoustic source, and/or that this source has a central location relatively to the head (e.g., in front of the face of the person). It may additionally or alternatively, be detected that the person perceives the two sounds with the best possible clarity, has the best possible understanding of a speech, and/or other. Alternatively or additionally, it may be detected that the person perceives the quietest sound they can hear. Alternatively or additionally, it may be detected that the person perceives the loudest sound they can withstand. Alternatively or additionally, it may be detected that the person perceives a desired sound level, to assess a level of comfort.

This detection may be based on a proactive response of the person, e.g., the person presses a button, blinks their eyes, performs an expected movement, verbally communicates, etc.

Advantageously, it may be detected that the person no longer moves their head. The person naturally moves their head to improve their perception of the sounds, and when the person no longer moves their head, it may be surmised that the person achieved the best possible perception.

For example, if during a lapse of time the changes in the values of the at least one parameter representative of the head's position does not exceed a threshold (or respective thresholds), then it is considered that the person no longer moves their head.

This lapse of time may be set to a fixed value, e.g., a value between 1 second and 30 seconds. Alternatively, the method may comprise a step of determining the lapse of time to be used to detect that a person no longer moves their head. This determination may be based on the person (e.g., on the experience of the person, is the person familiar with audiometry tests, on the age on the person, etc.), on the environment (e.g. on the loudness of the environment, for example it may be surmised that it's easier to achieve best perception in a quiet environment or in an acoustically simple environment with little or no echoes, and thus the lapse of time in a quiet/simple environment may be shorter than in a noisy/acoustically complex environment), and/or on other criteria.

Upon receipt of an indication that the person has achieved the best possible perception of the sounds, the position of their head and/or ears may be measured, e.g., using the same means as for the repeated measurements such as gyroscopes, etc., or using different means. The result(s) of this measurement may be stored as the current value(s) from which the sensitivity and/or the latency shift of the person is/are calculated.

Alternatively, upon receipt of an indication that the person has achieved the best possible perception of the sounds, the last value(s) of the parameter(s) measured at step (c) may be stored as the current value(s) from which the sensitivity and/or the latency shift of the person is/are calculated.

The current value(s) stored at step (e) may thus be of the same type as the monitored values of the at least one parameter representative of the person's head position, or of a different type(s). In particular, the measures based on which the audio signal(s) is/are adapted may be more accurate than the measure(s) of the best perception position.

For example, when the user moves their head seeking for a better listening, the method may comprise measuring in real time two set of spherical coordinates, one for each ear, and adapting the audio signal accordingly, when it is detected that the perception is optimized, another parameter, e.g., a single azimuthal angle value indicative of the head orientation, may be measured and stored.

Once step (f) is executed, i.e., when the differential sensitivity and/or latency shift value(s) is or are calculated, the current value stored at step (e) may be removed from the memory, or not.

The method described above may be carried out by means of various hardware, e.g., a virtual reality and/or augmented reality headset, an audio headset, a headphone, a webcam, an infrared camera, a depth camera, earbuds, or earphones, e.g., connected to a smartphone, etc.

The invention is not limited to a particular type of audio signal. For example, the audio signals may comprise narrowband audio signals, e.g., pure tone audio signals, and/or broadband audio signals, e.g., audio signals representing speech sounds, music, environment noise sounds (e.g., street sounds, transportation sounds, forest sounds, plane motor, etc.), etc., or a combination thereof.

By “narrowband” is it meant here that the ratio of the variations in frequencies and a central frequency is smaller than 5%, advantageously than 1%.

By “broadband”, it is meant that a ratio of a difference between a maximum and a minimum frequency and a median frequency is strictly higher than 0.05, advantageously higher than 0.3, e.g., higher than 0.5. A broadband audio signal may for example occupy the 300-3400 Hz range, or even the whole audible range. Selecting a broadband audio signal for step (b) may allow achieving accuracy very quickly.

Advantageously, the method may further comprise receiving an overall volume command e.g., from a user interface or from a calculation based on at least one measured value of the parameter(s) representative of the head position and adapting the audio signals based on this received command. That is, the person may set a preferred overall volume, thus allowing assessing an overall sensitivity, based on such typical use.

The invention is not limited either to a specific relationship between audio signals and parameters representative of the head position.

In particular, the value(s) of the at least one parameter representative of the head position may impact the amplitude, e.g., a root-mean-square amplitude or any other loudness or volume parameter, and/or the phase of at least one audio signal.

Advantageously, the amplitude(s) of at least one of the two audio signals, advantageously of the two audio signals, may vary depending on the value(s) of this at least one parameter. Step (c) may comprise repeatedly adapting an amplitude of the at least one of the two audio signals based on the at least one measured value. This may allow assessing a differential sensitivity between the two ears.

For example, one of the audio signals has an overall amplitude, e.g., a root-mean-square amplitude, that does not depend on the azimuthal angle of the head, while the other audio signal that an overall amplitude, e.g., a root-mean-square amplitude, that varies linearly with the azimuthal angle.

Advantageously, the two audio signals may correspond to a single source (which may be a real source, in particular when the audio signals result from recordings, or a simulated source).

This source may be a spatially limited acoustic source, or an acoustically complex environmental source.

Advantageously, a phase shift between the two audio signals may vary depending on the value(s) of at least one parameter representative of the head position, e.g., a polar angle. This may allow assessing a latency shift, e.g., a phase shift between the two ears.

Advantageously, the audio signals may comprise broadband audio signals corresponding to speech, preferably in a language the person understands, preferably their mother tongue. The resulting latency shift may reflect a brain asymmetry as the Broca's area is located on a single hemisphere. This may be particularly interesting as it appears that the latency related to brain asymmetry may vary significantly from one person to another, depending on e.g., if the person is very right-handed, ambidextrous, quite left-handed, etc.

This may allow obtaining a better correction as it may be adapted to the person and to the type of sound, e.g., it may be detected that an actual audio signal corresponds to speech and correction factor values applied to left and/or right audio signal may be selected based on latency shift values previously obtained for this person with test audio signals corresponding also to speech.

Advantageously, steps (b)-(f) may be repeated with different audio signals, e.g., with pure tones of different frequencies and/or amplitudes. This may allow obtaining more information about the person's hearing abilities.

Advantageously, the audio signals may successively comprise audio signals of different types. At step (b), the audio signals may be of a first type, e.g., audio signals corresponding to speech, and once step (d), (e) or (f) is performed, steps (b)-(f) may be repeated with audio signals of a second type, e.g., audio signals corresponding to environmental sounds. The differential sensitivity values and/or latency shift values calculated with such different audio signals may be analysed, thus allowing a better assessment of the hearing abilities.

In an embodiment, the audio signals may first comprise narrowband audio signals, e.g., pure tone audio signals (respectively, broadband audio signals), and afterward (e.g. after a lapse of time, once it is detected that the person has achieved their best perception, once it is detected that the person has achieved their best perceptions over a number of different narrowband (respectively broadband signals), etc.), the audio signals may comprise broadband audio signals (respectively, narrowband audio signals).

In an embodiment, the audio signals may first comprise broadband signals representing speech sounds (respectively non-speech audio signals), and afterward (e.g. after a lapse of time, once it is detected that the person has achieved their best perception, once it is detected that the person has achieved their best perceptions over a number of different speech signals (respectively non-speech signals), etc.), the audio signals may then comprise non-speech audio signals (respectively broadband audio signals representing speech sounds). The non-speech audio signals may comprise narrowband audio signals or non-speech broadband audio signals, e.g., audio signals representing music, environmental sounds such as forest sound, motor sounds, etc. In this embodiment, comparing the latency shift value(s) obtained for audio signals representing speech sounds to the latency shift value(s) obtained for non-speech audio signals may allow assessing a delay possibly related to the brain asymmetry, given that the Broca's area is located on one hemisphere only of the brain.

For example, at step (c), a phase shift between the two audio signals may be adapted based on the measured at least one value of at least one parameter representative of the person's head position. There may be provided at a first step (b) narrowband audio signals (or broadband signal without speech signals) and at subsequent first step (f), a first latency shift may be calculated based on the current value of step (e) corresponding to a best perception while the audio signals comprised narrowband signals (or, respectively, speechless broadband signals). There may be also provided at a second step (b), which may be before or after the first steps (b)-(f), broadband audio signals, e.g., audio signals corresponding to speech, and at subsequent second step (f), a second latency shift may be calculated based on the current value of step (e) corresponding to a best perception while the audio signals comprised the broadband signals. The first latency shift may be interpreted as corresponding to a sum of possible latency shift due to audio hardware and latency shift due to varying left and right auditory canals. The second latency shift may be impacted by the brain dissymmetry, the Broca's area involved in speech recognition being located in the left hemisphere. That is, the second latency shift may be interpreted as a sum of possible latency shift due to audio hardware, latency shift due to varying left and right auditory canals and latency shift caused by the Broca's area being on the left hemisphere. Thus, a delay caused by this brain asymmetry may be estimated based on the first and second latency shifts, e.g., by calculating a difference between the second latency shift and the first latency shift.

In an embodiment, the sensitivity calculated at step (f) may be used to augment an existing audiogram.

Classical audiograms only typically include up to 11 measured points per ear. The method described above may thus provide additional measures by comparing known values with unknown values.

Alternatively, the sensitivity and/or latency shift calculated at step (f) may be used to create a new extended audiogram.

In an embodiment, the results of step (f), e.g., the calculated sensitivity and/or latency shift, may be used to correct further audio signals. A correction factor to be applied may be calculated from the results of step (f).

The method described above may be used to calibrate new audio hardware, or possibly the same audio hardware, in particular when there are changes in the environment. Measuring an individual's hearing abilities as described above, e.g., using head tracking linked to an application on a smartphone, may thus allow corrective measures to be applied to audio equipment to dramatically improve perceived clarity.

The method described above may be performed in typical environments, for example to calibrate a hearing head in a noisy environment the user is familiar with. Calibration may also take into consideration the type of audio signals.

The method may for example comprise storing a plurality of hearing profiles, each corresponding to a respective environment, e.g., a profile for listening sounds when in a bus, a profile for listening sounds at home, etc., and/or to a type of audio signals, e.g., signals corresponding to speech, music, etc. Users may thus calibrate any audio technology to match their hearing abilities and listening conditions.

In an embodiment, the method may further comprise measuring at least one value of at least one sound parameter representative of the environment, such as a parameter related to noise (e.g., a signal-to-noise ratio), a sound power level, a main frequency, etc.

The measured at least one value may comprise one single value, a relatively limited number of values, e.g., between 2 and 50 values, or a higher number of values, e.g., more than 50 values. In this latter case, the measured values may for example comprise a sound recording over a lapse of time, e.g., 10 seconds or 1 minute, a frequency spectrum obtained based on such a recording, etc.

The measured value(s) may also be obtained from a recording over a lapse of time. The measured values may for example comprise, for a limited number of frequencies corresponding to the highest peaks of the spectrum, e.g., 5 or 10 peaks, a frequency value and a peak amplitude value.

This measured at least one value of at least one sound parameter representative of the environment may be associated the value(s) obtained at step (f). For example, the values corresponding to the 5 or 10 highest peaks of the frequency spectrum measured from the environment may be associated with every differential sensitivity value and/or value of a latency shift of the person obtained for audio signals provided to the human person when she/he was in this environment.

This association may allow to determine a correction factor (to be applied to further audio signals while the human person is in another environment) taking into consideration this another environment. In particular, this can take into consideration the masking phenomenon. Also, relative sensitivities between high frequencies and low frequencies may vary depending on the environment. Adapting the correction to the environment may thus allow achieving a better sound perception.

The wording “another environment” encompasses both another location and a same location but at a different time, e.g., 3 hours later, 1 day later or even 1 second later.

When the human person is in an environment (said “another environment” here), at least one value of at least one sound parameter measured in said another environment, e.g., a sound pressure level value, or values corresponding to the 5 or 10 highest peaks of the frequency spectrum, etc., may be used to determine the correction factor to be applied.

In an example, this or these value(s) measured for this another environment, i.e., the environment the person is actually in, may be compared to a plurality of value(s) previously measured in various environments, so as to identify the previous environment that is the most similar to the environment the person is actually in. Once the most similar previous environment is determined, the method may comprise selecting the corresponding differential sensitivity value(s), value(s) of a latency shift of the person, an extended audiogram, correction factor value(s) and/or other result(s) from step (f) obtained for audio signals provided in this most similar previous environment, to determine the correction factor.

That is, there may be stored in a memory a plurality of set of values, each set of values corresponding to a previously tested environment and comprising:
At least one value of at least one sound parameter representative of this previous environment, such as a signal-to-noise ratio, a spectrum, etc.
At least one value obtained pursuant to executing steps (a)-(f) in this previous environment, such as differential sensitivity value(s) and/or value(s) of a latency shift, a correction curve, etc.

In the example described above, the method may comprise, when the human person is in an environment (said “another environment” here), at least one value of at least one sound parameter of this environment, and then comparing this measured value(s) to sound parameter values stored in the memory, so as to identify the best matching environment.

The invention is not limited to identifying a single best matching environment. For example, there could be determined a weighted combination of previous environments and the correction could be determined by applying such weighted combinations to corresponding corrections factors.

In an embodiment, data obtained from the previous tests may be provided to a model for training, e.g., to a machine learning model.

The machine learning model may for example be a linear regression model, a logical regression model, or other.

Associating the value(s) obtained at step (f) with the at least one value of the at least one sound parameter representative of the environment may comprise providing the value(s) obtained at step (f) and the at least one value of the at least one sound parameter representative of the environment to a model for training. When the person is in another environment and a correction factor is to be determined, the method may comprise measuring at least one value of at least one sound parameter of this another environment the person is actually in, and providing this measured at least one value to the trained model, for the trained model to output values allowing to determine the correction factor, e.g., differential sensitivity value(s) and/or value(s) of a latency shift, a correction curve, etc.

A neural processor comprising a neural processing unit arranged to execute machine learning algorithms, e.g., by operating on predictive models such as artificial neural networks or random forests, may be used to carry out the training and subsequent predictions.

In particular, the value(s) obtained at step (f) and the at least one value of the at least one sound parameter representative of the environment may be provided to a machine learning model, e.g., a deep learning model.

The method may thus comprise:
initializing a model, e.g., a machine learning model;
determining a plurality of set values, of each set of values corresponding to a previously tested environment and comprising at least one value of at least one sound parameter representative of this previous environment, and at least one associated value obtained pursuant to executing steps (a)-(f) in this previous environment;
generating a training feature vector for each set of values of the plurality of sets of values;
training the model using the training feature vectors.

Training feature vectors are thus obtained from previous executions of steps (a)-(f) and are used for training the model. Such supervised training may allow obtaining a mapping function between environments and correction factors—for this person.

Pre-existing libraries, such as PyTorch, CatBoost, TensorFlow or other known libraries may be used to assist implementation.

The method may further comprise when the person is in another environment:
measuring at least one value of at least one sound parameter of this another environment the person is actually in;
providing this measured value(s) representative of the actual environment as a correction factor request to the model, e.g., a machine learning model;
executing the model using the provided measured value(s) representative of the actual environment to determine a correction factor to be applied to this person in this actual environment.

The measured value(s) representative of the actual environment is/are thus used as input when executing the model to determine the correction factor value(s).

The correction factor may then be applied to audio signals provided to the user, for the user to achieve a high-quality perception of the corresponding sounds.

The invention is not limited by the number of previously tested environments. This number may be relatively small, e.g., in the range from 2 to 20, in particular when simple comparisons are carried out, e.g., 3 (for example a quiet environment, a normal environment and a noisy environment). This number may be higher, e.g., in the range from 5 to 1000, advantageously from 21 to 200, in particular when a machine learning model is trained.

Advantageously, when steps (a)-(f) are carried out, the results of step (f) may be associated with at least one value characterizing the audio signals of step (b). That is, there may be determined an audiogram for a first type of signals, e.g., audio signals corresponding to music, and an audiogram for a second type of signals, e.g., audio signals corresponding to speech, e.g., podcasts.

These audio profiles may be stored. In particular, the audio profiles may also take into consideration the environments, as described above.

When later the person wishes to hear sounds of a first type, e.g., a podcast, the correction factor values may be determined based on the associated audio profile, e.g., the audiogram for speech audio signals.

The invention in not limited by a particular method for selecting the correction factor values to be applied. For example, there may be a limited number of types of audio signals and a best match method may be carried out. In an embodiment, a model, e.g., a machine learning model may be involved, using for example training and prediction methods similar to the training and prediction methods described above to take into consideration the environment.

More generally, the method may comprise associating the results of step (f) with:
at least one sound parameter representative of the environment;
at least one time value, e.g., a time of the day (perception may vary depending on whether it's the morning, evening, etc.);
at least one parameter relative to the test audio signals, e.g., relative to the type of content (speech, music, pure tone, etc.), loudness (quiet sound or loud sound), rhythm, and/or other; and/or
other parameter(s).

The correction factor to be applied may thus be determined based on at least one value corresponding to the actual environment, time, audio signals, and/or etc., which may allow obtaining an accurate and high-quality correction. In particular, a model may be trained with the associated data and the trained model may predict a best correction factor.

The method described above may be utilised to measure all or any frequencies across the audible spectrum in any listening environment. It may be more accurate than a speech to noise test.

In terms of HRTFs (head related transfer functions), the method described above may be utilised to identify where an individual perceives a sound to emanate from, thus allowing generating custom sets of HRTFs.

The method described above may also be used when testing new audio hardware and functionality to give a balanced and consistent frequency response irrespective of external factors such as level of listening, background noise or dynamic control.

The method may be used for example to customize hearing aids, calibrate headphones and loudspeakers. It may allow compensating imperfections in audio hardware and listening environments along with establishing the effectiveness of binaural models for virtual reality and augmented reality technologies.

In an embodiment, the audio signals are themselves obtained by applying a correction on original audio signals, the correction being based on results obtained at a previous execution of the method. That is, the method may be executed a first time, possibly with various audio signals, and the results obtained at step (f), e.g. sensitivity values each corresponding to a frequency, may be applied before the method being executed a second time and/or at each real-time adapting of the audio signals.

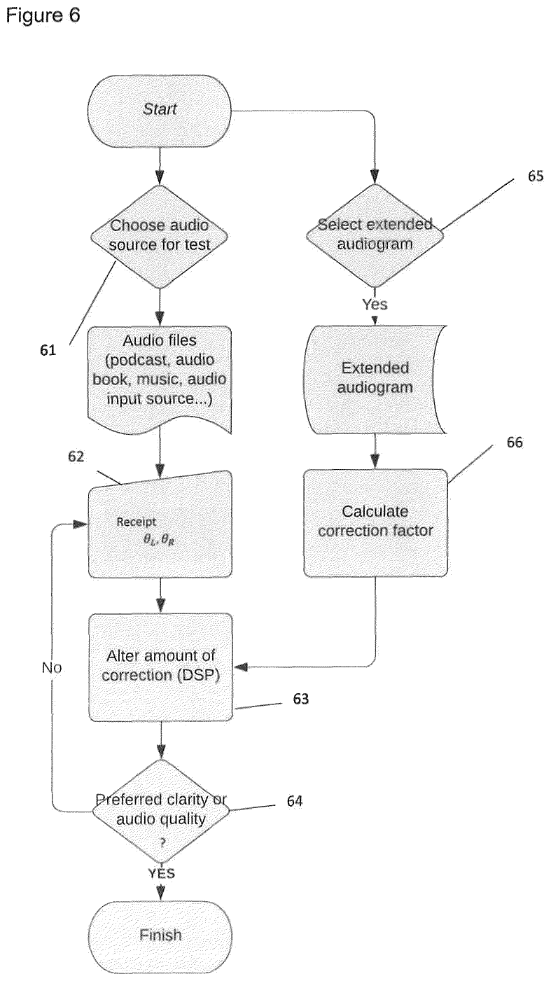

Thus, when the method further comprises utilising the sensitivity and/or a latency shift value(s) calculated at step (f) to determine a correction factor to be applied to further audio signals, it may also further comprise applying the correction factor to the further audio signals, transmitting the corrected signals to the ear speakers, and adapting the corrected signals in real time based on measured values of at least one parameter representative of the head position. This embodiment will be described more precisely with reference to FIG. 6 and FIG. 7C.

Advantageously, the method may further comprise utilizing a standard audiogram, e.g., an audiogram provided by standard ISO 7029-2017, as an initial audiogram based on which correction factor values are calculated and applied to the audio signals. The corrected signals are transmitted to the ear speakers and are adapted based on measured values of at least one parameter representative of the head position. That is, a standard audiogram, e.g., an expected audiogram given the age and/or gender of the person, is used as a starting point, e.g., during an initial calibration phase as illustrated by FIG. 7A.

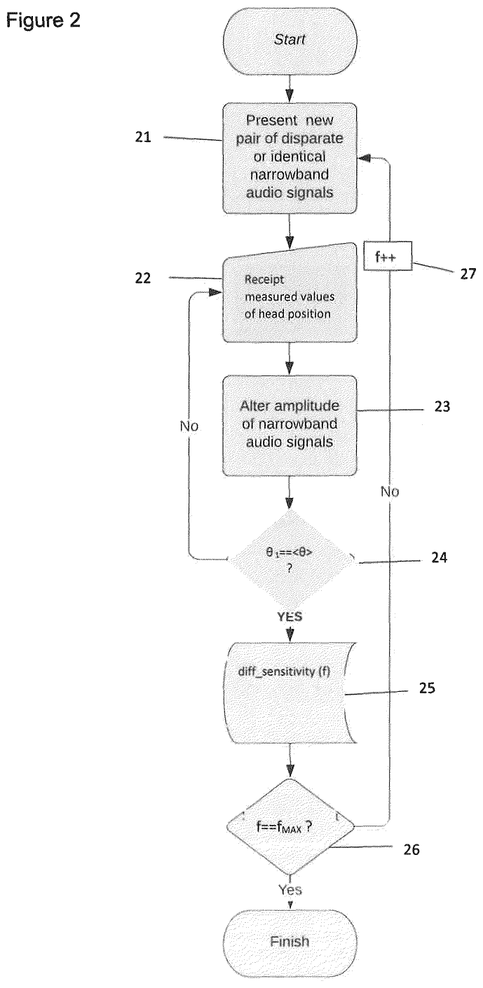

Thus, the method may comprise various phases, wherein steps (a)-(f) are carried out during each phase:
An initial calibration phase, wherein a standard audiogram is used to correct the audio signals, the percentage of correction varying based on the head position, and the position of best perception is used to adapt this standard audiogram, as illustrated in FIG. 7A;
A main calibration phase, with or without adaptation of the audio signals based on an audiogram, during which relative amplitude between left and right signals is adapted based on the head position, and wherein steps (a)-(f) allow to obtain an extended audiogram, as illustrated in FIGS. 2-5 and 7B; and/or
A final calibration phase, wherein the extended audiogram of the main calibration phase or of the initial calibration phase is used to determine correction factor values to be applied to audio signals, thus allowing to assess if this theoretical correction actually fits to the person, as illustrated in FIGS. 6 and 7C.

In an embodiment, these three phases may be carried out. Alternatively, only the initial calibration phase may be performed. Alternatively, only the main calibration phase is carried out. Alternatively, a single one of the initial and main calibration phases is carried out and the resulting extended audiogram is used to carry out the final calibration phase.

There is also provided a transitory or non-transitory computer readable medium storing computer executable code which when executed by a processor causes the processor to carry out at least one of the methods described herein above. The medium may be any entity or device capable of storing the program. For example, the medium can comprise a storage means, such as a ROM, for example a microelectronic circuit ROM, or else a magnetic recording means, for example a hard disk. An optical storage may also be used.

There is also provided a computer program product comprising instructions for carrying out at least one of the methods described herein above when the program is executed by a processor. These programs can use any programming language, and be in the form of source code, binary code, or of code intermediate between source code and object code such as in a partially compiled form, or in any other desirable form for implementing the methods according to the invention.

It is also provided a device for assessing hearing abilities of a human person having two ears, the device comprising processing means and transmitting means, wherein
the processing means are arranged to receive or generate two audio signals, each audio signal corresponding to a respective ear,
the processing means are also arranged to receive at least one measured value of at least one parameter representative of the person's head position and adapt the audio signals based on said at least one measured value (it is meant here that at most one of the two audio signals may be left as is);
the transmitting means are arranged to transmit the adapted audio signals to respective ear speakers for each ear speaker to convert the respective audio signal into a sound at the person's corresponding ear; and
wherein the processing means are arranged to
repeat the measured value reception and subsequent adaptation often enough for the sounds provided to the person to correspond to his/her actual head position;
detect that the person has found their best perception of the two sounds;
responsive to the detection, calculate from a current value(s) of the at least one parameter representative of the person's head position an overall sensitivity value, a differential sensitivity value and/or a latency shift value, e.g. an internal ear phase shift value.

There may for example be calculated an overall sensitivity value, in particular when adapting the audio signals comprising adapting the overall amplitude of the audio signals. The overall sensitivity value may correspond to the smallest amplitude the person can hear, the loudest amplitude the person can withstand, a level of comfort range, an optimum level of comfort, etc.

The transmitting means may also be arranged to transmit the calculated sensitivity and/or a latency shift, e.g., to displaying means or further processing means.

The device may further comprise receiving means to receive the audio signal(s) and/or the measurements upstream the processing means.

The device may for example comprise or be part of one or several processors, for example a digital signal processor (DSP), or another processor. In particular, the device may advantageously comprise or may communicate with a processor comprising a neural processing unit (NPU).

The device may be a circuit board or a processor for example.

The receiving means may for example comprise an input pin, an input port, a communication module, or other.

The processing means may for example comprise a CPU (for Central Processing Unit) core, or other.

The transmitting means may for example comprise an output pin, an output port, a communication module, or other.

The device may be part of a smartphone, a computer, or any other electronic apparatus such as an HMD, hearable or wearable.

There is also provided a system comprising the device described above, as well as the ear speakers.

The system may for example comprise:
position or motion sensors to be arranged on the head of the person, e.g., gyroscopes integrated into the ear speakers to monitor the head position;
a camera to capture a video of the head of the person, and further processing means to track movements of the head on the captured video; and/or
any other system capable of tracking heard rotation, such as a device based on LIDAR, RF, optical, inertial, mechanical, magnetic, or stretch, amongst others.

The system may for example comprise a camera, e.g., a webcam, and a memory storing a motion tracking program.

The invention is explained in further detail, and by way of example, with reference to the accompanying drawings wherein:

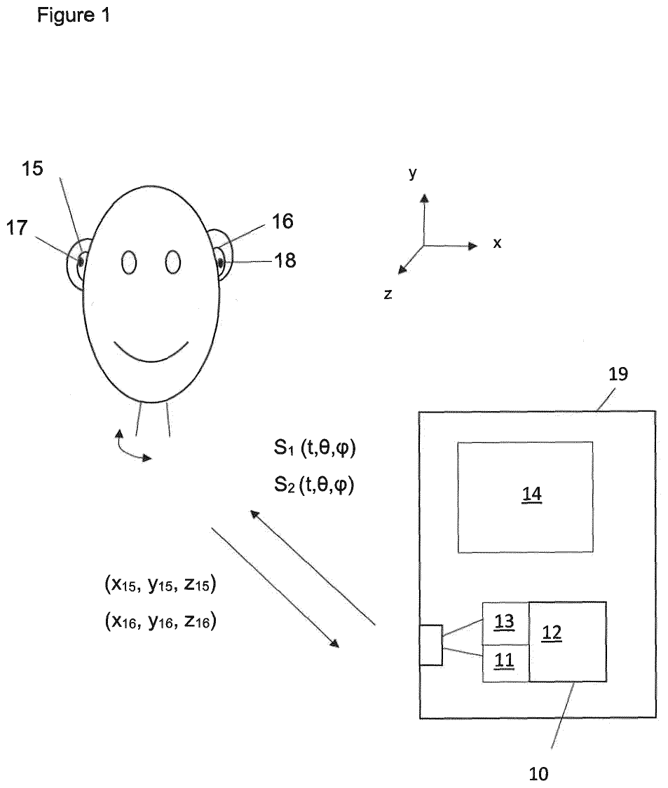

FIG. 1 schematically illustrates an example of a system according to an embodiment of the invention.

FIG. 2-7C schematically illustrate various examples of methods according to embodiments of the invention.

Referring now to FIG. 1, a device 10 according to an example embodiment of the invention comprises receiving means 11, e.g., input pins, processing means 12, e.g., a processor core, and transmitting means 13, e.g., output pins.

The illustrated device 10, e.g., a processor, may be part of an apparatus 19, e.g., a smartphone, a computer etc.

This apparatus 19 may also comprise displaying means, e.g., a screen 14.

The device 10 communicates, e.g., via Wi-Fi, Bluetooth, or another wire or wireless technology, with ear speakers 15, 16, e.g., earbuds.

The earbuds 15, 16 include integrated gyroscopes or MEMS accelerometers 17, 18. These accelerometers 17, 18 are adapted to measure values of parameters representing the position of the head of a person wearing the earbuds 15, 16, e.g., an azimuthal angle and a polar angle for each ear, or as illustrated in FIG. 1 Cartesian coordinates for ear.

The receiving means 11, may receive, e.g., from the Internet, audio signals for the earbuds 15, 16. Alternatively, these audio signals may be generated by the processing means.

The processing means adapt the audio signals, or at least one of the two audio signals corresponding to the two earbuds 15, 16, based on head position values measured by the accelerometers 17, 18.

The adapted signals are sent to the earbuds 15, 16.

The head position measurements and the adaptations as done rapidly enough for the person not to perceive it, e.g., every millisecond. The person will move their head to seek for the most comfortable hearing.

When the person has achieved their best perceptions, the person will naturally stop moving their head. This may be detected, and the current position may be measured and stored so as to calculate based on this best perception head position an overall sensitivity value, a differential sensitivity or a latency shift between the ears.

In a first embodiment, as illustrated by FIG. 2, the audio signals are narrowband audio signals, e.g., pure tone signals that may be synthetized by the processing means.

In this example, these two pure tone signals have a same frequency f.

This frequency may initially be a low frequency value, e.g., less than 100 Hz, e.g., 20 Hz or 30 Hz.

Initial audio signals are sent to the ear speakers at step 21. If the person perceives different sound amplitudes, he/she will rotate their head. Meanwhile, the position of the head is monitored (step 22) and the audio signals adapted accordingly (step 23).

For example, at step 22, the processing means received measured values of an azimuthal angle θ in the (x-z) plane of FIG. 1, i.e. the horizontal plane, normal to the gravity vector in this figure. This azimuthal angle θ may for example have a value equal to 0 degrees when the earbuds 15, 16 are in a plane parallel to the plane (x-y). Alternatively, the azimuthal angle θ may for example have a value equal to 0 degrees when the audio signals are initially sent to the person, i.e., we measure a change as compared to an initial position.

At step 23, the amplitude of one of the two pure tone signals, or of the two pure tone signals, e.g., a root-mean-square amplitude, may for example vary linearly with an azimuthal angle.

For example, while each audio signal may be written as:

$$∝ = A sin ( 2 π ft + ψ ) \qquad ( 1 )$$

amplitude parameter A of one of the signals may be written

$$A = {A}_{0} + b ( θ - {θ}_{0} ) \qquad ( 2 )$$

wherein A₀ and b are constants.

Steps 22 and 23 and repeated as long as the person moves their head, i.e., as long as the condition of test 24 is not reached. At step 24, it is detected if the head position has moved, e.g., by comparing the last measured value to an average over a lapse of time of the previously measured values. If it is detected that the head position has not really changed during this predetermined lapse of time, e.g., 5 seconds, then, the last measured values of the head position are stored, and a differential sensitivity is calculated based on these last measured values, at step 25.

For example, a person may hear the two sounds with equal amplitudes when their head is an angle θ_{eq_p} which may be different from another azimuthal angle θ_{eq_s} for which the two audio signals actually have equal root-mean-square amplitudes. At angle θ_{eq_p}, one of the audio signals may have an amplitude that is lower than the other, for example 5 or 10% lower. As the person perceives the two sounds with equal amplitudes, it can be surmised that the ear speakers have different conversion ratios and/or that the ears of the person have different sensitivity, for this frequency and this amplitude.

For example, the differential sensibility for the current frequency f may be calculated as

$$Δ = {S}_{R} ( \frac{{A}_{0} + b ( Θ - {Θ}_{0} )}{{A}_{L}} - 1 ) \qquad ( 3 )$$

Where, alleging that only the signal sent to the right ear has its amplitude that varies with angle θ according to (2), S_{R} is the right ear sensitivity, A_{L} the amplitude of the signal at the left ear.

Then, the processing means may calculate from an overall sensitivity and from the differential sensitivity, a sensitivity for the left ear and a sensitivity for the right ear.

To obtain the overall sensitivity, the user may set their desired level of listening, such that different profiles may be generated, thus compensating for equal loudness contours. This may facilitate a wider range of listening environments. In an alternative embodiment (not illustrated by FIG. 2), the overall amplitude, i.e., amplitude of both the left and right audio signals, may be adjusted based on a measured value of a parameter representing the head position, such as height of the head. For example, if the person moves their head downward, both signals become quieter, while if the person moves their head toward the ceiling, the audio signals become louder. This may allow assessing an overall sensitivity and/or a desired overall amplitude.

At step 26, it is detected whether there are sufficient differential sensitivity values, e.g., by comparing the frequency of the tone signals of step 21 to a maximum frequency f_{MAX}, e.g., 20 KHz. If the maximum frequency is not reached yet, a new frequency value is obtained—this is schematically illustrated by the incrementing step 27, and the processing means synthetize at step 21 new tone signals having this new frequency.

Steps 21 to 26 are thus repeated for a number of frequency values, e.g., 10 or 20 frequency values, within the range of 20 Hz-20 kHz. These frequency values may be part of a predefined list. The skilled person will understand that at step 27, the current frequency value may be replaced by the following frequency value in the list.

In a not represented alternative embodiment, steps 21 to 26 may also be repeated with audio signals having varying amplitudes, for example once with low amplitude audio signals, another time with medium amplitude audio signals, and a third time with loud audio signals.

This may be explored with wide dynamic range signals as well, which may allow achieving an appropriate compromise. Alternatively, a microphone may monitor SPLs (sound pressure levels) in real-time and adjust accordingly.

Once sufficient data points are obtained, that is, test 26 is achieved, an extended audiogram, which may also be repeated for phase, showing the variations of the obtained right and left sensitivity values with frequency may be displayed, e.g., on a screen.

In an alternative example, the audio signals of step 21 are still pure tone signals, but with two different respective frequencies.

The amplitudes may initially be the same for the two audio signals, and the amplitude(s) may then vary depending on the head position.

For example, a first audio signal is a pure tone at 1 kHz, with an initial amplitude of −24 dBFS, and a second audio signal is a pure tone at 8 kHz, with an initial amplitude of −24 dBFS. The first audio signal corresponds to the right ear and the second audio signal correspond to the left ear. If the head rotates to the left, the amplitude of the second signal decreases, and if the head rotates to the right, the amplitude of the first audio signal decreases.

When the sounds are perceived as being of equal loudness, the person may indicate that balance has been achieved, e.g., by no longer moving their head.

It may be noted that the relative difference in sensitivity between the two signals has been established within defined listening conditions. The method may be repeated with different combinations of signals, e.g., as often as the person wishes. Each time the method is repeated, the profile becomes more accurate at representing what each unique listener experiences sonically.

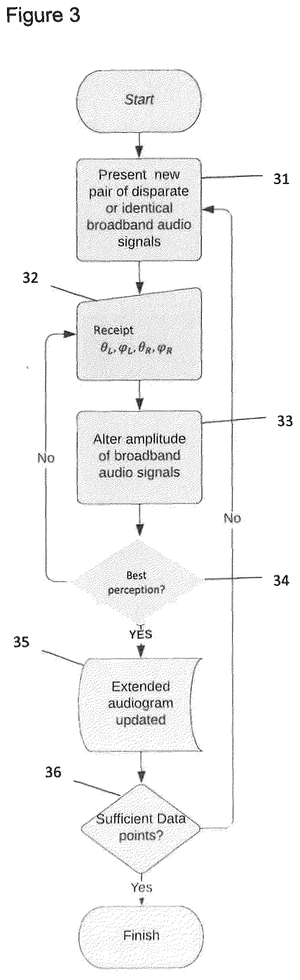

In a second embodiment, as illustrated by FIG. 3, the audio signals of step 31 are broadband audio signals within the range of 20 Hz-20 kHz, e.g., speech signals, music signals, sound effects signals etc.

The audio signals, which may be disparate or identical, are sent independently to the corresponding earphones.

There may be provided a not illustrated step, comprising asking the person via a user interface to set the overall volume such that he/she could understand the speech, or distinguish the instruments or the sounds. This may also be achieved by rotating the head to control the volume until optimal clarity is achieved.

Then, the audio signals are sent with the amplitude of the internal harmonics varying at step 33 with the head rotation measured at step 32.

For example, if the signals may be written as the following i indexed sum, i varying from 1 to a n value:

$${α}_{R , L} = \sum {A}_{i , R , L} sin ( 2 π {f}_{i} t + {ψ}_{i} )$$

Amplitude parameters Aᵢ for the left and right ears (respectively) may vary with the head rotation, e.g., linearly. It may be detected at step 34 whether equal loudness, maximum clarity or the auditory field being centred is achieved, e.g., by detecting that the person no longer moves their head. Then a difference in sensitivity is calculated based on this position of the head, and an extended audiogram is updated at step 35.

Steps 31 to 35 may be repeated if the overall number of differences in sensitivity values is too low, i.e., if test 36 is not achieved.

For example, a first speech signal, e.g. with estimated frequency response of left ear (this may be based on the results of narrowband tests, as described in FIG. 2, or by referring to standard ISO7029:2017 and cross referencing with the person's age and gender if the person wishes to skip out the previous stages), is played in the left ear at (for example) −24 dBFS, and a second speech signal, e.g., with estimated right frequency response, is played in the right ear initially at (for example) −24 dBFS. If the head rotates to the left the effect of estimated spectral sensitivity corrections between the two signals decreases, and if the head rotates to the right then the effect of the spectral sensitivity corrections increases. When the signals are perceived as being of maximum clarity and/or located in the centre, the user indicates that balance has been achieved. The relative difference in sensitivity between the two signals is then established within the defined listening conditions.

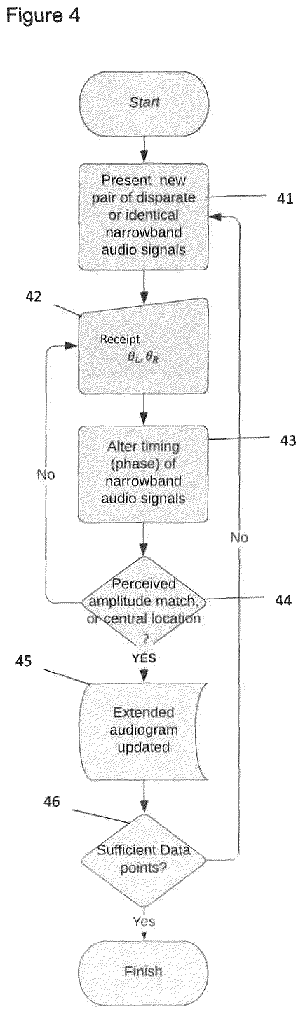

In a third embodiment, as illustrated by FIG. 4, the audio signals are two narrowband signals, and their phase vary with the head position, e.g., such that a phase shift between the audio signals is reduced as the person rotates their head toward a virtual source.

The signals of step 41 may be two disparate or identical audio signals within the audible range. These signals are sent independently to respective earphones. Phase (timing) of the signals is adapted at step 43 based on measured values of parameter(s) received at step 42, these parameter(s) representing the head position.

If the person indicates that sounds are perceived as optimum loudness or equal loudness (that is, in the centre of the auditory field), i.e., if a condition of test 44 is reached, then a latency shift may be calculated, and an extended audiogram may be updated at step 45.

Steps 41 to 45 may be repeated if the overall number of calculated latency values is too low, i.e., if the condition of test 46 is not reached.

For example, 1 kHz tone at −24 dBFS is played in the left ear, with a 1 kHz tone at −24 dBFS played in the right ear. If the head is rotated to the left the signal on the left is delayed by increasing amounts in milliseconds and/or the signal on the right moves forward in time; and if the head rotates to the right, then the right signal is delayed in an identical manner and/or the signal on the left moves forward in time. The phase of the left/right signal may for example vary linearly with the azimuthal angle. The proportionality coefficient may be chosen depending on an amount of correction required.

This technique will identify phase/timing anomalies due to hearing abilities or reproduction technologies.

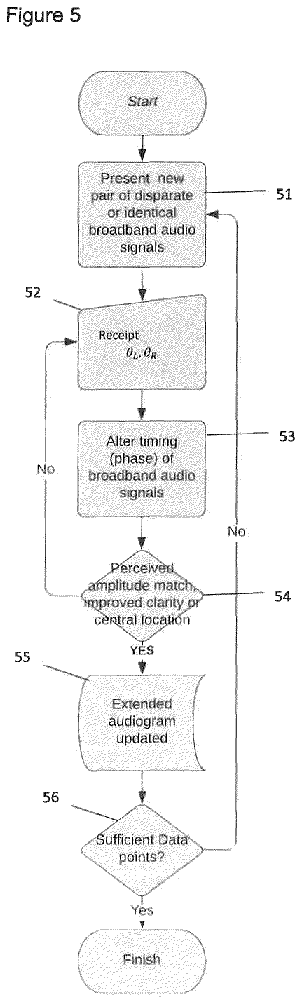

In a fourth embodiment, as illustrated by FIG. 5, the audio signals are initially, i.e., at step 51, two disparate or identical broadband signals. The phase(s) of these signals is adapted at step 53 based on measured head rotation values received at step 52, and these monitoring and adapting are repeated as long as the condition of test 54 is not reached. It may be detected at test 54 that the person perceives the two sounds with optimal loudness or maximal clarity, or equal loudness or originating from a source at the centre of the auditory field.

For example, complex alert sounds may be sent to each ear, with varying timing differences within specified frequency ranges. Moving the head to left or right alters the relationship between the timing differences until the signal appears to be in the centre and/or clear.

Then, an extended audiogram may be updated, at step 55, based on a position of the head the user had when the condition of test 54 is reached.

In the embodiment of FIG. 6, the results of a previous execution of the method described above, e.g., an updated extended audiogram, are used at the adapting step.

At step 61, an audio file is selected, e.g., a podcast, music, etc.

At step 65, an extended audiogram is selected.

For example, several audiograms may be stored, each audiogram corresponding to a type of environment (street, forest, bus, quiet room, noisy room, plane, etc.). It may be detected that the person is in a determined type of environment, e.g., in the street, and the corresponding audiogram may be selected.

At step 66, a correction factor is calculated based on the selected audiogram.

At step 62, values of the person head position are received.

At step 63, the left and right sound signals are recalculated, taking into consideration both the measured values of the head position obtained at step 62 and the correction factor obtained at step 66.

At non-illustrated steps, a value of sensitivity and/or latency shift is recalculated based on the position of the head corresponding to the optimal perception, i.e., upon achievement of test 64.

The selected audiogram may possibly be corrected accordingly, as well as the correction factor.

In fine, this method may allow limiting the correction factor depending on whether the person is satisfied by the correction or finds the correction factor too unfamiliar.

That is, based on the results of any one of previously described embodiments with reference to FIGS. 2 to 5, it may be computed an audio correction factor. This auditory correction factor is then applied to a broadband audio signal of the user's choice, which varies with head rotation allowing users to choose their preferred percentage of the computed audio correction factor for ongoing use.

For example, an ambulance driver wears headset in vehicle whilst being driven and rotates head whilst listening to speech transmitted over the radio, indicates when clarity is maximised without inducing ear fatigue.

Referring now to FIG. 7A, there is illustrated an example method for initial calibration.

A human person is provided with two ear speakers, one for each ear.

A step 701, an expected audiogram is selected based on the age and gender of this person. The expected audiogram may be a standard one for this age and gender, e.g., it may be selected referring to standard ISO 7029-2017.

The method is not limited by the way the age and gender information are obtained. For example, the method may comprise not illustrated age and gender values receiving steps. Alternatively, there may be stored a gender and a date of birth for the person.

At step 702, a correction factor is calculated from the audiogram selected at step 701. The correction factor may comprise a number of values, e.g., between 8 and 1096, each value corresponding to a frequency.

At step 703, an audio file is selected, e.g., a podcast, music, etc.

At step 704, values of the person head position are received. For example, at each time sample, threes values, for the respective Cartesian coordinates x, y, z, are received.

Referring to these FIGS. 7A-7C, x and y coordinates correspond to a plane normal to the direction of the gravity vector, while z corresponds to this direction.

At step 705, the left and right sound signals are recalculated, taking into consideration both the measured values of the head position obtained at step 704 and the correction factor value(s) obtained at step 702. For example:
depending e.g., on x value, the frequency correction of the left and right corrected sound signals is adjusted; for example, if the person moves toward the left, no correction is applied, and if the person moves toward the right, for each frequency, a percentage of the corresponding correction factor value is applied to this frequency. When 100% correction is applied no further correction is applied if the person goes on beyond the corresponding displacement (e.g., 1 metre at the right, or 90 degrees clockwise). Alternatively, if the person goes beyond this position corresponding to 100% correction, further correction may be applied, e.g., up to 150% of the correction factor values; and optionally:
the phase of the left signal relative to the right signal is adjusted based on for example the received y value (corresponding to backward and forward movements).
the overall amplitude of both left and right audio signals is adjusted based e.g., on the received z value. For example, if the person head moves downward, both audio signals become quieter, and if the head moves upward, both signals become louder.

When test 706 is achieved, i.e., when it is detected that the person has achieved their position of best perception, the current values of x, y, z are stored. At step 707, there are calculated:
sensitivity values based on the stored values of x and z;
a latency shift value based on the stored y value.

Although this is not illustrated, steps 703-707 may be repeated with various audio files, thus allowing to generate an extended audiogram.

A step 708, at least one value of at least one sound parameter representative of the environment is measured, e.g., a sound pressure value, a FFT frequency spectrum, etc.

At step 707, the calculated sensitivity and latency shift values are associated with the results of this measuring step 708. When steps 703-707 are repeated a number of times so as to generate an extended audiogram, the extended audiogram can be associated with the results of this measuring step 708.

The method of FIG. 7A can be carried out in various environments. For each environment, there may be stored a set of values comprising an extending audiogram (corresponding to this person and this environment), and the results of this measuring step 708.

Referring now to FIG. 7B, it illustrates a method of main calibration that in an embodiment can be carried out once the initial calibration is over. Alternatively, no initial calibration is carried out.

I is first selected at step 710 whether the audio signals will be narrowband audio signals or broadband audio signals.

If broadband is selected, at step 711, two broadband audio signals are provided to the two respective ears of the person.

Head orientation is monitored at step 712, and the broadband audio signals are adapted accordingly at step 713. For example:
the relative amplitude between left and right signals may be adjusted based on the x values, e.g., if the user moves toward the left, the left audio signal may become louder as compared to the right audio signal, and if the person moves toward the right, the left audio signal becomes quieter as compared to the right audio signal;
the overall amplitude may be adjusted based on the z value, as described with reference to step 705 of FIG. 7A;
the phase shift may be adjusted based on the y value, as described with reference to step 705 of FIG. 7A.

At test 714, it is determined whether best perception is achieved. If not, the method goes back to step 712. When the best perception is achieved, current values of coordinates x, y, z are stored and used to update the extended audiogram, at step 715.

At step 716, it is detected if the number of updated points in the extended audiogram reached a threshold. If not, the method goes back to step 710.

If pursuant to step 710, the audio signals are to be narrowband audio signals, at step 717, two narrowband audio signals are provided to the two respective ears of the person. These two narrowband audio signals may have a same frequency, or different frequencies. These two narrowband audio signals may have an initial phase shift, or not.

Head orientation is monitored at step 718, and the broadband audio signals are adapted accordingly at step 719, e.g., as described with reference to step 713.

At step 720, it is tested whether the person perceives the two sounds with equal loudness. If not, the method goes back to step 718.

When it is detected that the signals are perceived with equal loudness, and/or that best perception is achieved, current values of coordinates x, y, z are stored and used to update the extended audiogram, at step 721.

At step 722, it is detected if the number of updated points in the extended audiogram reached a threshold. If not, the method goes back to step 710.

Further, at least one value of at least one sound parameter representative of the environment is measured at step 723, e.g., a sound pressure value, a FFT frequency spectrum, etc.

This measured value(s) may be associated with the updated extended audiogram.

If one of the tests 716, 722 has a positive outcome, then the method described with reference to FIG. 7C may be carried out. In an alternative embodiment, only the initial and final calibration may be carried out.

At step 731, the extended audiogram updated at steps 715, 721 of FIG. 7B (or of step 707 of FIG. 7A) is selected and correction factor values are calculated accordingly at step 732.

Audio files can be selected at step 733.

The corresponding audio signals are provided to the ears of the person. Head position is monitored at step 734.

At step 735, the correction factor values are applied to the audio signals, and then the corrected audio signals may be adjusted based on the measured values representative of the head position.

At step 736, it is detected whether the person has achieved their best perception.

If the outcome of test 736 is positive, the extended audiogram of step 731 is updated based on the actual values of the x, y, z coordinates, at step 737.

Thus, it may be detected that the person is actually not satisfied when a theoretical correction is applied and somehow wishes to mitigate this correction.

Further, at least one value of at least one sound parameter representative of the environment is measured at step 738, e.g., a sound pressure value, a FFT frequency spectrum, etc.

This measured value(s) may be associated with the updated extended audiogram. A set of values comprising the values of the updated extended audiogram (pursuant to the execution of steps 733-737 a number of times, e.g., 10 or 100 times so as to cover the whole audio spectrum) and the values measured at step 738 may be provided to a machine learning model. The machine learning model is thus trained with a relatively accurate feature vector.

1. A method for assessing hearing abilities of a human person having two ears, the method comprising:
(a) providing two ear speakers to the person, one for each ear;
(b) for each ear speaker, providing an audio signal to the ear speaker for the ear speaker to convert said audio signal into a sound at the person's corresponding ear;
(c) measuring at least one value of at least one parameter representative of the person's head position, adapting at least one of the two audio signals based on the at least one measured value, and repeating the measuring and adapting often enough for the sounds provided to the person to correspond to their actual head position;
(d) detecting that the person has found their best perception of the two sounds,
(e) responsive to the detection, storing at least one current value of at least one parameter representative of the person's head position; and
(f) calculating from the at least one stored value of the at least one parameter representative of the person's head position a differential sensitivity value and/or a value of a latency shift of the person.

2. The method of claim 1, wherein step (c) comprises repeatedly adapting a phase shift between the two audio signals based on the at least one measured value.

3. The method of claim 1, wherein the audio signals comprise broadband signals.

4. The method of claim 1, wherein steps (b)-(f) are repeated with different audio signals.

5. The method of claim 4, wherein the different audio signals comprise broadband signals representing speech sounds and audio signals representing non-speech sounds, respectively, the method further comprising:
calculating at step (f) a first latency shift from a current value obtained at step e) while the audio signals comprised broadband signals representing speech sounds;
calculating at step (f) a second latency from a current value obtained while the audio signals comprised audio signals representing non-speech sounds; and
estimating from the first latency and the second latency a delay caused by brain asymmetry.

6. The method of claim 1, wherein at step c):
the at least one parameter representative of the person's head position comprises at least a first and a second parameters (y, z), and
repeatedly adapting the at least one of the two audio signals based on the at least one measured value comprising repeatedly adapting a first parameter, e.g. an amplitude, of the at least one of the two audio signals based on the at least one measured value of the first parameter (y) and repeatedly adapting a second parameter the at least one of the two audio signals distinct from the first parameter, e.g., a phase shift between the two audio signals, based on the at least one measured value of the second parameter (z).

7. The method of claim 6, wherein at step c),
the at least one parameter representative of the person's head position comprises a third parameter (x), the first, second and third parameters forming coordinates of a coordinate system; and wherein,
repeatedly adapting the at least one of the two audio signals based on the at least one measured value comprising repeatedly adapting a third parameter of the at least one of the two audio signals based on the at least one measured value of the third parameter, the third parameter being distinct from the first and second parameter.

8. The method of claim 1, further comprising utilising the value(s) calculated at step (f) to determine a correction factor to be applied to further audio signals.

9. The method of claim 8, further comprising applying the correction factor to the further audio signals, transmitting the corrected signals to the ear speakers, and adapting the corrected signals in real time based on measured values of at least one parameter representative of the head position.

10. The method claim 8, further comprising:
measuring at least one value of at least one sound parameter representative of the environment;
associating the value(s) obtained at step (f) with the at least one value of the at least one sound parameter, for the correction factor to be applied to further audio signals while the human person is in another environment to be determined based on at least one value of at least one sound parameter measured in said another environment.

11. The method of claim 10, wherein associating the value(s) obtained at step (f) with the at least one value of the at least one sound parameter representative of the environment comprises providing the value(s) obtained at step (f) and the at least one value of the at least one sound parameter representative of the environment to a machine learning model for training.

12. The method of claim 1, wherein step (c) comprising repeatedly adapting amplitude of the at least one of the two audio signals based on the at least one measured value.

13. A computer readable medium storing computer executable code which when executed by a processor causes the processor to carry out the steps of claim 1.

14. A device for assessing hearing abilities of a human person having two ears, the device comprising processing means and transmitting means, wherein;
the processing means are arranged to receive or generate two audio signals, each audio signal corresponding to a respective ear;
the processing means are also arranged to receive at least one measured value of at least one parameter representative of the person's head position and adapt the audio signals based on said at least one measured value; and
the transmitting means are arranged to transmit the adapted audio signals to respective ear speakers for each ear speaker to convert the respective audio signal into a sound at the person's corresponding ear,
wherein the processing means are arranged to:
repeat the measured value reception and subsequent adaptation often enough for the sounds provided to the person to correspond to his/her actual head position;
detect that the person has found their best perception of the two sounds; and
responsive to the detection, calculate a differential sensitivity and/or an latency shift value from a current value(s) of at least one parameter representative of the person's head position.

15. A system comprising the device of claim 14, the ear speakers, preferably at least one of:
a camera to capture a video of the head of the person, and further processing means to track movements of the head on the captured video, and
position or motion sensors to be arranged on the head of the person.
